(19) United States
(12) Patent Application Publication
(10) Pub. No.: US 2008/0081942 A1
(43) Pub. Date: Apr. 3, 2008

(54) SYSTEMS FOR HEART TREATMENT

(75) Inventors: Suresh PAI, Mountain View, CA (US); Nicanor DOMINGO, Santa Clara, CA (US)

Correspondence Address: LEVINE BAGADE HAN LLP, 2483 EAST BAYSHORE ROAD, SUITE 100, PALO ALTO, CA, 94303, US

(73) Assignee: Bay Innovation Group, Mountain View, CA (US)

(21) Appl. No.: 11/746,429

(22) Filed: May 9, 2007

Related U.S. Application Data
(63) Continuation of application No. 10/990,361, filed on Nov. 15, 2004, now abandoned.
(60) Provisional application No. 60/519,915, filed on Nov. 14, 2003.

(51) Int. Cl. A61N 1/362; A61N 1/05

(52) U.S. Cl. 600/16; 607/129; 607/9

(57) ABSTRACT

Devices and methods for treating degenerative, congestive heart disease and related dysfunction are described. Passive and active cardiac support structures mitigate changes in ventricular structure (i.e., remodeling) and deterioration of global left ventricular performance related to tissue damage precipitating from ischemia, acute myocardial infarction (AMI) or other abnormalities. Cardiac efficiency is improved by providing reinforcement that restores or maintains an elliptical ventricular shape and mimics the position and positive inotropic effects of helical wound myofibrils to provide active contraction of the ventricle in synchrony with the metabolically required cardiac pace or output. In addition, the cardiac support structures compensate or provide therapeutic treatment for congestive heart failure and/or reverse the remodeling that produces an enlarged heart. The structures may be implanted in target heart regions using less invasive surgical techniques, such as those involving port access or small incisions into the thoracic cavity.

### CROSS REFERENCE TO RELATED APPLICATIONS

This application claims the benefit of co-pending Provisional Patent Application Ser. No. 60/519,915, filed Nov. 14, 2004 and entitled, “Minimally Invasive Systems for Heart Constraint and Reshaping with Passive or Active Contraction” which is incorporated by reference herein in its entirety.

### FIELD OF THE INVENTION

The present invention relates generally to minimally invasive, mechanical, medical devices for treating or preventing congestive heart failure and related or concomitant vascular dysfunction. More specifically, the invention relates to cardiac support structures that mitigate changes in the ventricular and/or atrial structure and geometry and deterioration of global left and right ventricular and atrial performance related to tissue damage from myocardial ischemia, acute myocardial infarction (AMI), valve related disease or dysfunction, vascular related dysfunction, or other instigators of deterioration of cardiac output and/or function.

### BACKGROUND

Congestive heart failure (CHF) is a progressive and lethal disease if left untreated. The CHF syndrome often evolves as a continuum of clinical adaptations, from the subtle loss of normal function to the presence of symptoms refractory to medical therapy. While the exact etiology of the syndrome that causes heart failure is not fully understood, the primary cause of CHF is the inability of the heart to properly and adequately fill or empty blood from the left ventricle (i.e., left ventricular dysfunction) with adequate efficiency to meet the metabolic needs of the body.

In addition, non-cardiac factors can also be activated due the overall degenerative cycle that ensues. These include neuro-hormonal stimulation, endothelial dysfunction, vasoconstriction, and renal sodium retention all of which can cause dyspnea, fatigue and edema rendering patients unable to perform the simplest everyday tasks. These types of non-cardiac factors are secondary to the negative, functional adaptations of the ventricles, cardiac valves or load conditions applied to or resisted by these structures. Even with novel pharmacological, surgical and device-based therapies, symptoms can be alleviated, but the quality of life remains significantly impaired and the associated morbidity and mortality of the disease is exceptionally high.

Ischemic heart disease is currently the leading cause of CHF in the western world, accounting for greater than 70% of cases worldwide. In these cases, CHF can precipitate from ischemic conditions or from muscle damage (i.e., AMI due to obstruction of a coronary artery) which can weaken the heart muscle, initiating a process known as remodeling where changes in cardiac anatomy and physiology include ventricular dilatation, regional wall motion abnormalities, decreases in the left ventricular ejection fraction and impairment of other critical parameters of ventricular function. This left ventricular dysfunction is further aggravated by hypertension and valvular disease in which a chronic volume or pressure overload can alter the structure and function of the ventricle. Decreases in systolic contraction can lead to cardiomyopathy, which further exacerbates the localized, ischemia damaged tissue or AMI insult into a global impairment leading to episodes of arrhythmia, progressive pump failure and death.

Analogous to aneurysms in diseased hearts accompanying abnormally thin and weak myocardial tissue, ischemia-damaged and/or infarct damaged heart muscle tissue results in progressive softening or degeneration of cardiac tissue. These ischemic and infarcted zones of the heart muscle wall have limited, if not complete loss of tissue contractile functionality and overall physical integrity. Also, the disease is usually associated with a progressive enlargement of the heart as it increases contractility and heart rate in a compensatory response to the decreasing cardiac output. With this enlargement, the heart's burden is increased to pump more blood with each pump cycle. A phenomenon known as myocardial stretch is implicated in the cyclic feedback loop that causes areas of compromised heart muscle tissue to bulge outward. When the bulging is related to AMI, this behavior is characterized as infarct expansion. With this bulging, the heart's natural contraction mechanism is dissipated into and attenuated resulting in a marked and progressing decrease in cardiac output.

Normal cardiac valve closure (especially that of the mitral valve) is dependent upon the integrity of the myocardium, as well as that of the valve apparatus itself. The normal mitral valve is a complex structure; consisting of leaflets, annulus, chordae tendonae, and papillary muscles and any damage or impairment in function of any of these key components can render a valve structure incompetent. Impairment of valve function, due to independent factors (i.e., a concomitant valve pathology) or dependent factors (i.e., valve dilation related to dilated cardiomyopathy or mitral regurgitation due to atrial enlargement), can result in valvular insufficiency further exacerbating the degenerative CHF cycle.

The major objectives of heart failure therapy are to decrease symptoms and prolong life. The American Heart Association guidelines suggest that the optimal treatment objectives includes means to increase survival, exercise capacity, improve of quality of life, while decreasing symptoms, morbidity and the continued progression of the degeneration. Various pharmacological and surgical methods have been applied both with palliative and therapeutic outcome goals, however there still remains no cure for the condition.

Modern pharmacological approaches such as diuretics, vasodilators, and digoxin dramatically lessen CHF symptoms and prolong life by mitigating the non-cardiac factors implicated in the syndrome. Furosemide (more commonly known as Lasix) is also a valuable diuretic drug which eliminates excess water and salt from the body by altering kidney function and thereby increasing urine output thereby relieving the circulatory congestion and the accompanying pulmonary and peripheral edema. Vasodilators, like angiotensin-converting-enzyme (ACE) inhibitors have become one of the cornerstones in treatment of heart failure. These kinds of vasodilators relax both arterial and venous smooth muscle, thereby reducing the resistance to left ventricular ejection. In patients with enlarged ventricles, the drug increases stroke volume with a reduction in ventricular filling pressure. Digoxin has also been found to be positively inotropic (i.e., strengthens the heart's contraction capability).

On the surgical front, cardiomyoplasty is a recently developed treatment of CHF, where the latissimus dorsi muscle is removed from the patient's shoulder, wrapped around the heart and chronically paced in synchrony with ventricular systole with the goal of assisting the heart to pump during systole. The procedure is known to provide some symptomatic improvement, but is controversial with regard to its ability to enable active improvement of cardiac performance. It is hypothesized that the symptomatic improvement is primarily generated by passive constraint and mitigation of the degenerative, remodeling process. In spite of the positive outcome on relieving some of the symptoms, the procedure is highly invasive, requiring access to the heart via a sternotomy, expensive, complex and of unknown durability (due to the muscle wrap blood flow requirements and fibrosis issues). Another surgery of interest is an innovative procedure developed by R. Bautista, MD. In this procedure, the overall mass, volume and diameter of the heart are physically reduced by dissection and removal of left ventricular tissue. Besides being a highly invasive, traumatic and costly procedure, the actual volume reduction results in a reduction in valve competence and elicits the associated regurgitation. An alternative to this approach as also been proffered by surgeon, V. Dor MD. The Dor procedure provides surgical exclusion of akinetic and dyskinetic portions of the ventricle, reshapes the ventricle with a stitch that encircles the transitional zone between contractile and non contractile myocardium, and uses a small patch to reestablish ventricular wall continuity at the level of a purse string suture. Experience with the procedure has led to further refinements and enhanced clinical understanding of the benefits of this surgery. The principal benefits have been identified as diminished ventricular volume without deformation of the clamber and optimization of the ventricular shape to the preferred anatomical geometry. Normal myocardial fiber are known to be oriented in a spiral direction from the base of the heart to the apex with two opposite layers and well defined intersecting angles (per Bennington-Goertler, Vol. II). As such, this double spiral muscle fiber orientation facilitates a mere 30% of fibril shortening to output a 60% or greater ejection fraction. In dilated hearts resultant of the heart failure cascade the ventricle assumes a more spherical shape and this spiral architecture and hence the associated contractile efficiency is lost. In addition, the dilated ventricle also malpositions the subvalvular apparati. The papillary muscles tend to be displaced toward the lateral wall and thereby lose their normal orientation towards the apex eliciting retraction of the posterior leaflet, loss of leaflet coaptation and ultimately functional mitral regurgitation. Surgical treatment of this valvular dysfunction also includes a wide range of open procedure options ranging from mitral ring annuloplasty to complete valve replacement using mechanical or tissue based valve prosthesis. While being generally successful and routine in surgical practice today, these procedures are also costly, highly invasive, and are still have significant associated morbidity and mortality.

More recently, mechanical assist devices which act as a bridge to transplant such as the left ventricular assist device (LVAD) or the total artificial heart (TAH) implant have become available. LVAD's are implantable, mechanical pumps that facilitate the flow of blood from the left ventricle into the aorta. The latest, TAH technologies feature many improved design and material enhancements that increase their durability and reliability. However, the use of such devices is still limited by high costs and a lack of substantial, clinical evidence warranting their use.

Other device-based options for this patient subset include reshaping, reinforcement and reduction of the heart's anatomical structure using polymeric and metallic bands, cuffs, jackets, balloon/balloon-like structures or socks to provide external stress relief to the heart and to reduce the propensity/capability of the cardiac tissue to distend or become continually stretched and damaged with progressive pump cycles. Examples of such devices are United States Patent Application No. 2002/0045799 and U.S. Pat. No. 5,702,343. In addition, devices are being studied that attempt to prevent the tissue remodeling using tethers and growth limiting struts or structures described in various patents (e.g., U.S. Pat. No. 6,406,420).

In general, all of these concepts support the cardiac muscle and restrict growth externally and globally via surgical placement about the epicardium and in some instances are positioned across the cardiac muscle tissue. As such, these types of approaches require unnecessary positioning of the devices over healthy (non local, undamaged) areas or zones of the heart affecting the entire organ when the primary treatment is usually focused is on the left ventricle or the mitral valve annulus. This non-localized treatment can elicit iatrogenic conditions such as undesired valvular dysfunction or constrictive physiology due to over restriction of the heart by such restraints.

Recently, several device based options have also been introduced where implants are positioned by minimally invasive means in the coronary sinus in one configuration and then assume a post deployment configuration that constricts around the annulus to improve valve competence in dilated cardiomyopathy (e.g., United States Patent Application No. 2002/016628). The clinical efficacy of this approach while appealing is unknown at this time.

Finally, the ultimate treatment for people suffering end stage CHF is a heart transplant. Transplants represent a massive challenge with donor hearts generally in short supply and with the transplant surgery itself presenting a high risk, traumatic and costly procedure. In spite of this, transplants present a valuable, albeit limited, upside increasing life expectancy of end stage congestive heart failure patient from less than one year up to a potential five years.

It is evident that there is currently no ideal treatment among the various surgical, pharmacological, and device based approaches to treat the multiple cardiac and non-cardiac factors implicated with the syndrome of CHF. There is a clear, unmet clinical need for technology that is minimally invasive (ideally percutaneous) which can prevent, treat or reduce the structural remodeling to the heart and it's sub-structures across the continuum of the syndrome beginning acutely with the ischemia or ischemic infarct through the end stages where there is often left ventricular and valvular dysfunction refractory to conventional treatments.

Accordingly, there is a need for improved systems and devices to passively or actively improve cardiac output, reduce wall stresses, reinforce the walls, and reduce/limit volume of the heart muscle as required using percutaneous, minimally invasive surgical (MIS), and open surgical means or a combination thereof. Ideally, such a device could facilitate operator controlled “tailoring” of treatment using various embodiments of the invention at various chosen target zones (i.e., ventricles, atria, aorta, pulmonary artery, etc.). The custom tailoring of each system could serve a dual purpose of wall reinforcement/restraint of dilation, but also provide active compression to provide a potential positive inotropic effect.

Patients suffering from severe CHF, who are unresponsive to medication, are generally precluded from open surgical approaches and potentially awaiting transplant could derive massive and direct benefit from a minimally invasive treatment for their condition. The present invention offers such a treatment.

### SUMMARY OF THE INVENTION

Devices and methods according to the present invention not only offer an approach to limit further degeneration of CHF, but variations of the invention can also actively and/or passively facilitate positive or reverse remodeling (i.e., to provide a mild compressive force against the dilated ventricle in synchrony with the pace established by the A-V node) to induce pulsatile contraction of these structures to facilitate improved cardiac output and efficiency. As such, the subject devices and methods provide a potential, palliative or therapeutic response to the referenced disease state.

Variations or embodiments of the present invention provide cardiac support structures that offer structural rigidity and resistance to overdilation of the cardiac muscle fiber while maintaining an ideal, efficient ventricular shape, orientation of these support structures in specific anatomical positions similar to and in order to restore the helically would native myocardial fiber locations, and application of an energy source to provide active contraction of the myocardium in synchrony with metabolic and functional needs established by the pacemaker driving the electrical activity within the heart.

A benefit of these cardiac support structures is that they may work in concert to simultaneously provide reinforcement against myocardial stretch (or infarct expansion) and provide an active, positive inotropic during systole. Such devices and associated methods provide dynamic support or reinforcement. Further, they are active throughout the cardiac cycle—unlike previous device-based approaches that solely attempt to passively reduce the stress in the heart wall during diastole. Diastolic compliance can also be regulated or controlled with structures according to the present invention.

Though not necessarily the case, the cardiac support structures of the invention are typically implanted/deployed using a minimally invasive surgical approach. In practice, the subject structures can be placed via a sub-xiphoid approach which allows sufficient exposure and visualization of the heart using standard minimally invasive means to facilitate placement and anchoring of the support structure(s) at targets zones about the heart.

### BRIEF DESCRIPTION OF THE DRAWINGS

Certain aspects of the figures diagrammatically represent the present invention, while others may be indicative of preferred relations. Variation of the invention from what is shown in the figures is contemplated.

FIGS. 1A and 1B show perspective views dramatizing a healthy heart in systole and diastole respectively. FIGS. 1C and 1D show perspective views dramatizing a diseased (enlarged) heart in systole and diastole respectively. FIGS. 1E and 1F show a perspective views of a passive cardiac support structure embodiment on a new infarcted and a progressively, enlarged heart facilitating efficient, restored ventricular shape and alignment with natural helical myofibril orientation.

FIGS. 2A, 2B and 2C show a bottom view and two perspective views of a heart illustrating the preferred, helical fiber orientation. FIG. 2D shows the heart of FIG. 2B with a helical cardiac support structure superimposed in the same helical geometry.

FIGS. 3A and 3B show perspective views of two cardiac support structure embodiments. FIG. 3C shows a perspective view of the clip that anchors and interconnects the cardiac support structure embodiments in FIGS. 3A and 3B. FIG. 3D shows a perspective view of the cardiac support structure and clip of 3A, 3B and 3C components prior to interconnection. FIGS. 3E to 3H show perspective and detailed, close-up views of various interconnected cardiac support structure components from FIGS. 3A, 3B and 3C.

FIG. 4 shows a perspective view of the cardiac support structures and interconnected area in FIGS. 3A to 3H positioned upon and secured to the surface of a heart.

FIG. 5A shows a perspective view of an alternative cardiac support structure embodiment. FIG. 5B shows a heart with multiple, independent cardiac support structures, in FIG. 5A, deployed and secured in a helical pattern about the surface of the left ventricle.

FIGS. 6A to 6E show perspective views of a chest with heart, ribs and sub-xiphoid access incision illustrating the step-by-step positioning, release and securing of cardiac support structures in a helical pattern to the left ventricle.

FIG. 7 shows a perspective view of a heart with cardiac support structures of the invention secured to the left ventricle in an alternative helical pattern.

FIGS. 8 and 9 show a perspective views of a heart with cardiac support structures secured in helical patterns around the left and right ventricles.

FIGS. 10A and 10B show a perspective view and a cross-sectional view, respectively, of a heart with cardiac support structures secured in a helical pattern around the left ventricle.

FIGS. 11A and 11B show a perspective view and a cross-sectional view, respectively, of a heart with a cardiac support structure placed and secured such that the papillary muscles in the left ventricle are repositioned.

FIGS. 12A and 12B show perspective views of two hearts with multiple cardiac support structures placed and secured such that the papillary muscles in the left ventricle are repositioned.

FIGS. 13A and 13B show perspective views of two hearts with cardiac support structures placed and secured to reposition the papillary muscles in the left ventricle relative to the base of the heart at the atrial-ventricular groove.

FIG. 14 shows a perspective view of a heart with cardiac support structures placed and secured to reposition the apex of the heart relative to the papillary muscles in the left ventricle.

FIG. 15 shows a perspective view of a heart with multiple, independent cardiac support structures placed and secured to reposition the papillary muscles relative to each other within the left ventricle, the base at the atrial-ventricular groove, and the apex of the heart.

FIG. 16A shows a partial side-sectional view of a flexible, cardiac support structure that incorporates one or more wire with electrodes exposed to enable multi-site pacing to provide active contraction. FIG. 16B shows a perspective view of the electrodes in the support structure of FIG. 16A. FIG. 16C shows a perspective view of a heart with the active, cardiac support structure in FIG. 16A placed and secured in a preferred, helical pattern about the left and right ventricles and attached to an energy source.

FIGS. 17 and 18 show perspective views of hearts with two alternative cardiac support structures that incorporate electrodes for multi-site pacing.

FIGS. 19A and 19B show side views of a cardiac support structure embodiment in an extended and compressed and configuration, respectively, that utilizes alternative energy sources for active contraction. FIGS. 19C and 19D show schematic illustrations of application of electrical current as the energy source input to the cardiac support structure element in FIGS. 19A and 19B in order to allow active contraction or to induce expansion of the support structure element.

### DETAILED DESCRIPTION

Having described the characteristics and problems of congestive heart failure in the background and summarized hereto, the treatment method and apparati of the present invention will now be described in detail below. The variations of the invention described below may be used to provide a complete, comprehensive solution to treating congestive heart syndrome, and the contributing or associated co-morbid, anatomical, and physiological deficiencies. Addressing the multiple factors that affect or cause congestive heart disease can retard or reverse the implicated remodeling thereby treating or mitigating the congestive heart disease and associated symptoms.

With respect to these multiple factors the following applications are discussed in detail: Muscle Fiber Helix Restoring Cardiac Support Structures, Papillary Muscle Repositioning, Active Cardiac Support Structures and Integrated Multi-Site Pacing, and Cardiac Support Structures with an Integrated Active Compression Mechanism. In connection with these completer or partial solutions, various cardiac support structure components, deployment approaches and structure materials and general fabrication methods for the devices are described. Naturally, it is the intent that sometimes these solutions may be applied in a stand-alone fashion, and other situations in which any of the solutions will be utilized in any combination together for combined effect.

Before further discussion of the invention, however, it is to be understood that it is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “and,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

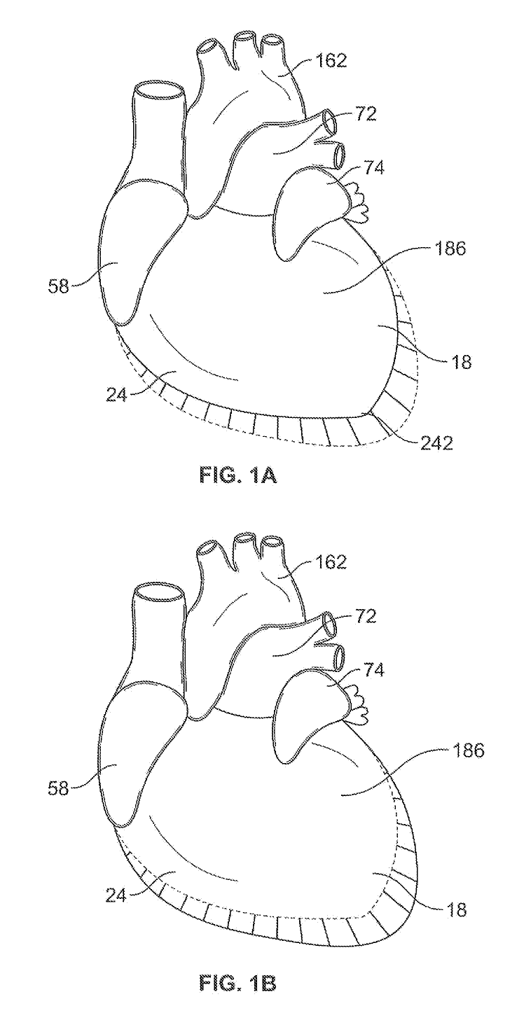

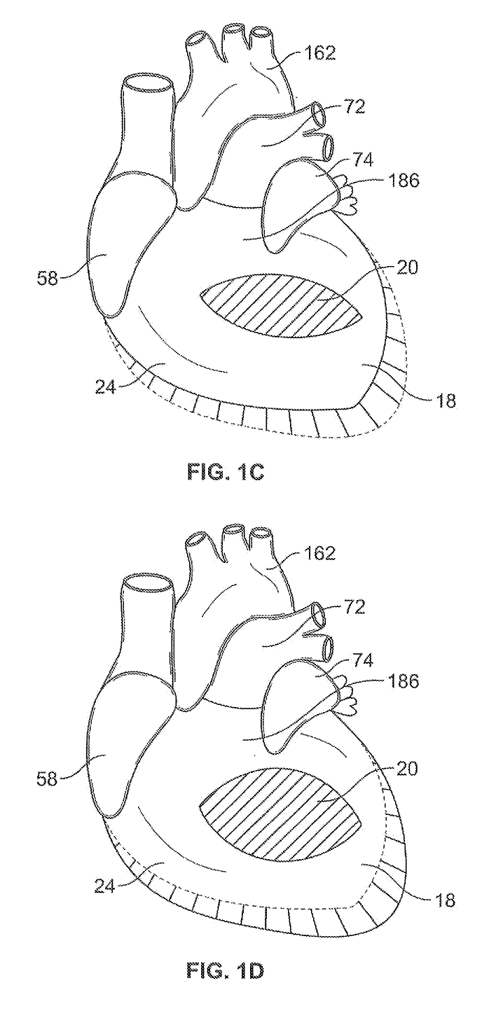

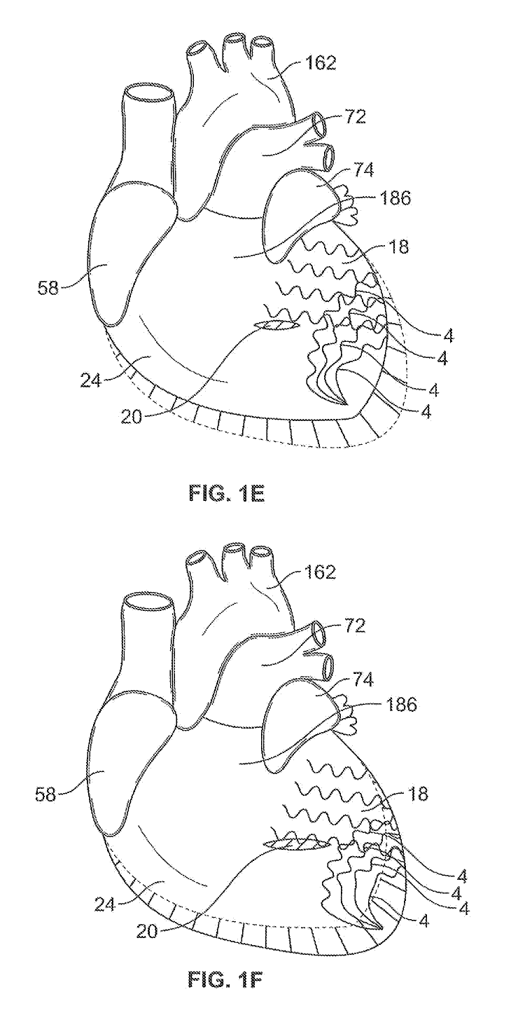

With initial reference to FIGS. 1A and 1B, an anterior view of a healthy heart in systole and diastole respectively is shown with directional arrows showing motion of the heart 186. The preferred evolutionary anatomical shape of the left ventricle 18 assumes an elliptical form positioned in an oblique orientation from the base to the apex 242. This ventricular shape (in stark contrast to the spherical shape typically presented in later stage heart failure patients) provides tangible benefits in contractile efficiency and valvular function by maintaining a desirable orientation relative to the helical wound myocardial fiber bundles and a desirable position of the subvalvular apparati.

In FIGS. 1B and 1C, perspective views are shown of a diseased (enlarged) heart in systole and diastole respectively. The infarcted or ischemic region 20 is shown to stretch from systole to diastole consistent with the progressive remodeling that occurs due to increased diastolic filling pressures exerted on the diseased tissue. A radial and axial expansion that is experienced by the heart leads to degenerative remodeling and concomitant organ enlargement. This enlargement can be localized along the anterior wall of the left ventricle 18, can be located or extend septally, can include the right ventricle 24, and/or can involve the mitral valve annulus 108. This remodeling is exacerbated and accelerated by deterioration of associated anatomic structures such as the atria 74 and 58, the aorta 162, the pulmonary artery 72, etc. which aid cardiac output in normal hearts by pulsating and augmenting the pumping action of the heart ventricles alone. Accordingly it is evident that it would be desirable to maintain or at least restore the benefits of the preferred elliptical shape of the left ventricle 18 and to take advantage of the helical fibril bundle orientation in compromised hearts.

FIGS. 1E and 1F show perspective views of a diseased heart reinforced with a cardiac support structure 4 of the invention. These cardiac support structures 4 restore the preferred helical fiber orientation, and are secured in position on the surface of the heart to limit myocardial stretch or infarct expansion by locally reinforcing the infarcted/ischemic regions 20 or other diseased sections of tissue, and limiting the tension applied to the tissue regions 20 in conjunction with diastolic filling pressure exerted directly against this diseased section. In this example, a cardiac support structure 4 is shown deployed and secured to the left ventricle 18 in a helical pattern to restore the natural, healthy cardiac fiber orientation.

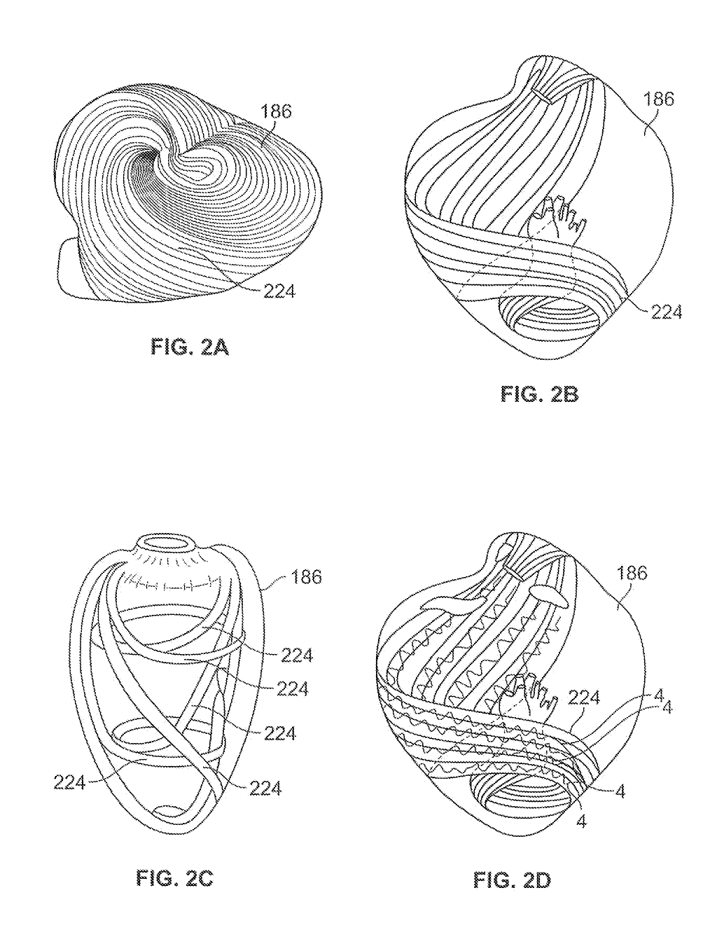

FIGS. 2A to 2C show the natural helical myocardial fiber orientation 224 of a normal heart. The helical orientation involves the left 18 and right 24 ventricles and can be completely unraveled into a flat sheet. During unwanted remodeling of the heart associated with congestive heart failure, the myocardial fibers unwind as the heart enlarges thereby compromising efficient contractility and wall motion usually enabled by the natural helically oriented myocardial fiber orientation 224. FIG. 2D shows a cardiac support structure embodiment of the invention designed to restore the myocardial fiber orientation by compressing the heart wall back into a helical shape. The compression applied by the structure depicted in the illustration can be instantaneously adjusted during intraoperative procedures, or can be adjusted over a period of time including post-discharge from the hospital.

Cardiac support structure aspects of the present invention comprise—individually, or in combination—components or devices including tensile member(s), anchor member(s) and deployment device(s). These components or devices are designed to be able to work alone or in concert in order to facilitate and provide palliative or therapeutic cardiac reinforcement in the following critical target areas of the heart: 1) papillary muscles; 2) cardiac valve annulus; 3) epicardium; 4) apex of the heart; 5) ventricular septum; and/or 6) myocardium. The sub-sections broken-out below will further describe treatments addressing corresponding specific aspects of the invention.

### Cardiac Support Structure Components

Many of the embodiments described below incorporate a tensile member terminating at anchor mechanisms at each end. The embodiments described below are adapted or configured to be positioned into or through the myocardium and define anchor mechanisms augmented by the inherent structure and deployment process and/or can incorporate one or more anchors to aid in positioning and securing the cardiac support structures in place.

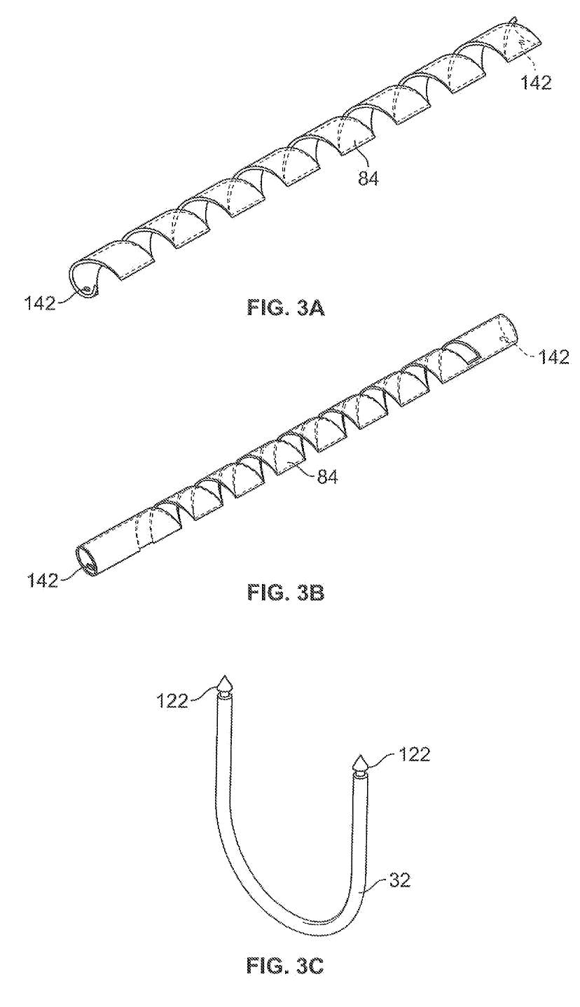

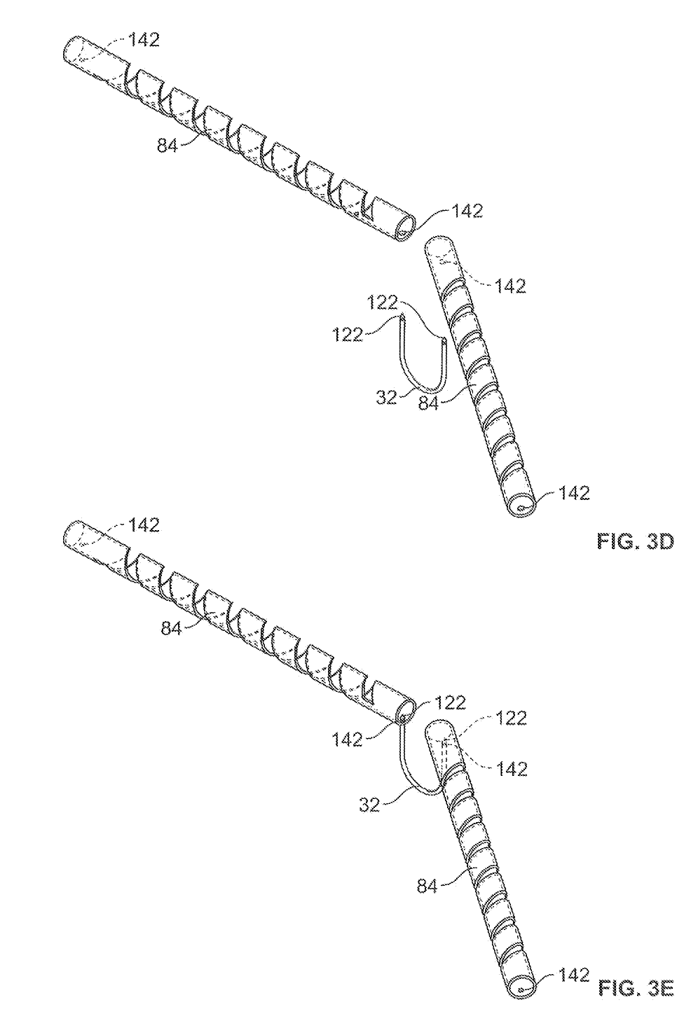

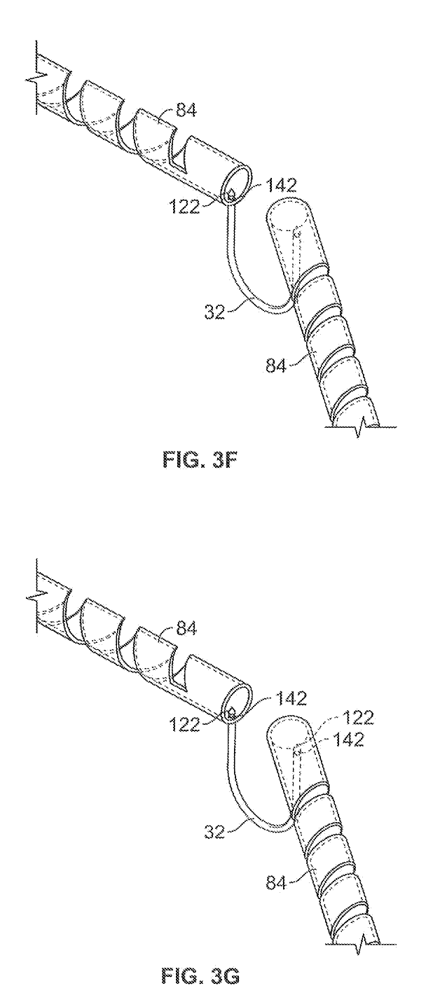

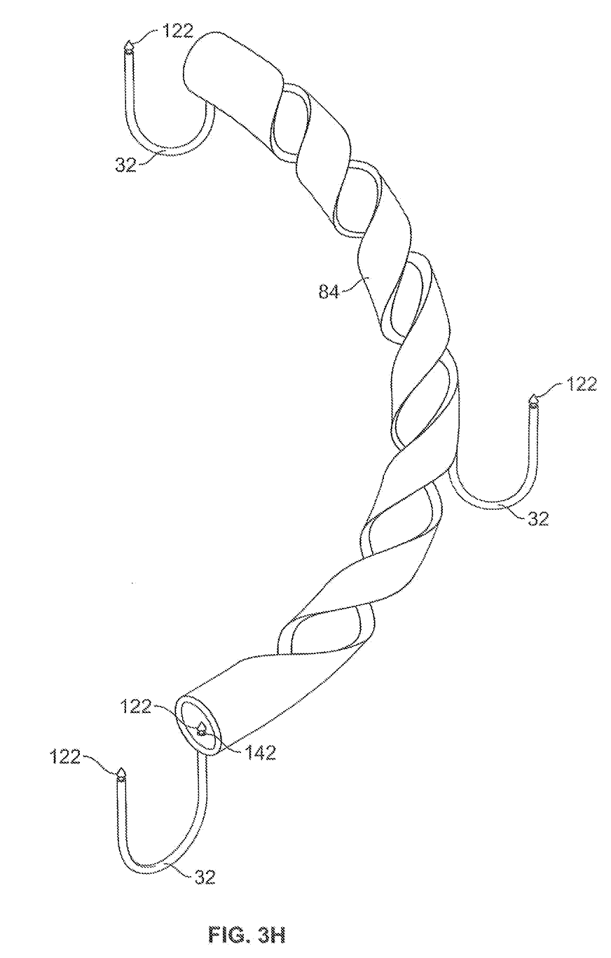

FIGS. 3A to 3H show a cardiac support structure embodiment 84 that incorporates anchors 32 separately deployable from the tensile members. It should be noted, however, that the anchors can be advantageously be integrated and/or interconnected with the tensile members. FIG. 3C shows an anchor 32 that can be inserted through myocardium and incorporates notches 122 that fit in mating openings 142 of the tensile members to secure it firmly in position for chronic use throughout the necessary cyclic life of the device. The tensile members shown in FIGS. 3A and 3B incorporate a spring component capable (if desired) of a pre-defined extension and contraction such that the tensile member 84 can be expanded during positioning and apply a compressive force against the heart to continuously urge and maintain the heart into the preferred helical orientation or any other orientation as defined by the operator during the implant procedure.

Tensile members 84 may comprise a tubing of raw material (metal, alloy, polymer, etc.) cut into a helical spring. It should be noted that other tensile member configurations can be used including solid wire or tubing, mesh members, standard wound coil springs or other geometrical patterns that define the degree of elasticity, and rigidity.

Once the anchors are positioned, the tensile members such as those shown in FIGS. 3A and 3B can be deployed locally and strategically arranged to provide the desired reinforcement and/or repositioning clinically desirable end effects. FIGS. 3D and 3E show the engagement of the openings in the tensile members shown in FIGS. 3A and 3B to the anchor shown in FIG. 3C. Once the opening of the tensile member 84 is placed over the end of the anchor, the anchor end locks to the tensile member firmly securing the tensile member to the anchor. As shown in FIGS. 3F to 3H, tensile members can be connected to anchors at the ends of the tensile members, at the mid-region of the tensile members, or anywhere where a suitable anchoring opening along the length of the tensile member is located.

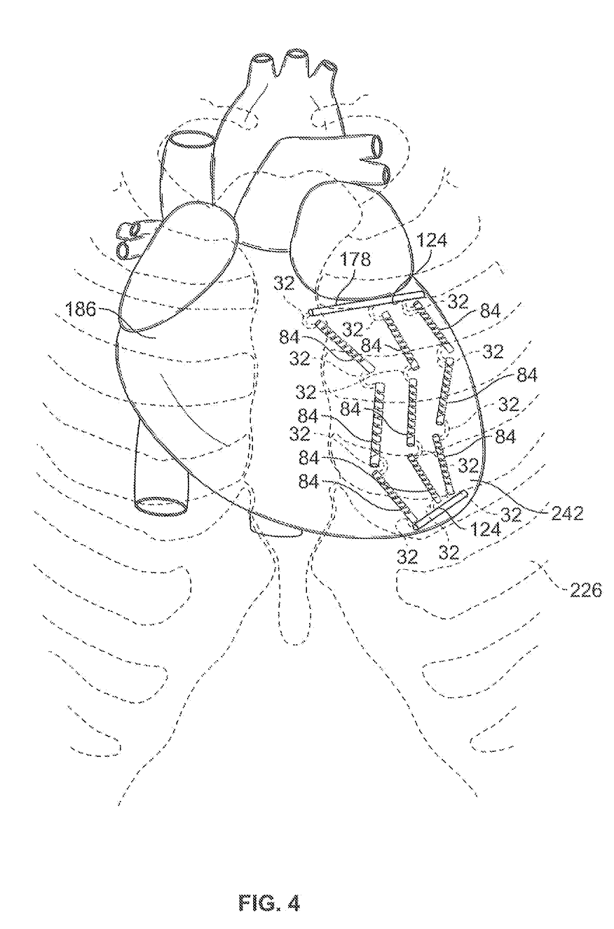

FIG. 4 shows a heart with an alternative cardiac support structure pattern utilizing the tensile members and anchors in FIGS. 3A to 3H described above with ribcage 226 superimposed over the subject system. In this embodiment, the cardiac support structures extend from the atrial-ventricular groove 178 to the apex of the heart 242 to maintain, facilitate or restore a desirable anatomical shape and relative positioning of heart subcomponents. At each of the extreme positions of the structure (i.e., adjacent atrial-ventricular groove 178 and apex 242), the anchors 32 are shown interconnected with locking anchor elements 124, the purpose of which is to stabilize the structure by capturing the anchors. In the alternative, individual caps (integrated with or interfacing with) the end anchors may be provided in the variation of the invention.

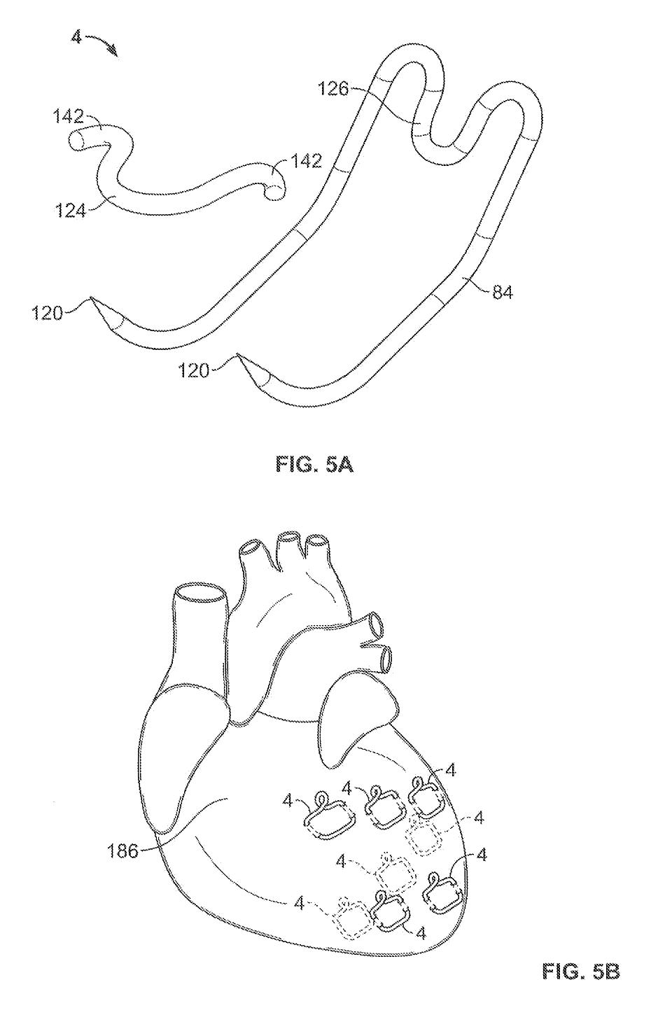

FIG. 5B shows cardiac support structures 4 as shown in FIG. 5A deployed through the myocardium of heart 186. In the cardiac support structure embodiment 4 in FIG. 5A, a single tensile member 84 is shown with spaced apart tissue penetration ends 120 on one side and a connecting spring on the opposite side 126. Upon insertion of the tissue penetrating ends 120 of the tensile members 84, a cardiac support structure anchor 124 can be placed over these ends to secure the member to the tissue surface or the ends of the tensile member can be tied to produce an axially-oriented tightening of the support structure 4. Still further, one free end of the tensile member can be subsequently inserted through myocardium at a spaced apart location to produce a three-dimensional, cinching effect.

According to one aspect of the invention, multiple cardiac support structures are secured to the heart tissue to produce a helical pattern as shown in FIG. 5B, thereby maintaining or repositioning the myocardial fibers into a helical pattern. The array of tensioning structures may thereby maintain or restore a more optimal pattern of myocardial fiber contraction.

It is this array, assemblage or pattern of spring elements that comprises an aspect of the invention; so too do the methods of selecting the points/regions for positioning the tensioning members, the methods of emplacing the same, and even the methods of their operation once emplaced.

Actually, the tensioning structures shown in FIGS. 5A and 5B are described in co-pending U.S. Patent Application No. 2003/0078465, entitled, “Systems for Heart Treatment,” to Pai et al. incorporated by reference herein in its entirety for any purpose. Many of the tensioning structures shown therein may be placed and used according to the present invention. Under such circumstances where known tensioning structures are involved, the invention concerns the use to which the known devices are put. Still, another aspect of the present invention involves the new tensioning structures disclosed herein.

In any case, FIG. 5B, illustrates an aspect of the invention involving the helical placement of the tensioning structures as the units spiral around the whole of the heart muscle (not just a surface patch) in support of the underlying spiraling myocardial fibers 224 as illustrated in FIG. 2D. In this regard, support structures 4 on the opposite side of the heart from that facing the viewer are indicated in dashed line.

Further details as to the helical placement of support structures is provided below. Before such discussion, however, some treatment is given to the manner in which the devices can be emplaced.

### Deployment of Cardiac Support Structures

Delivery systems can be used to deploy the cardiac support structures via a thoracotomy, thoracostomy, sub-xiphoid access 228, median sternotomy or other surgical access. In this manner, a deployment system 230 can access the heart along the epicardium (or endocardium) and position the cardiac support structures 4 at the desired locations in/on the heart. The delivery systems can be used to insert the anchors 32 (e.g., the embodiment shown in FIG. 3C) of the cardiac support structures 4 into or through myocardium 34 where they engage the myocardium, the epicardium, or the endocardium and tensile members 84 to attach the cardiac support structures to the heart. Once the anchors are positioned, the tensile members 84 such as those shown in FIGS. 3A and 3B can be arranged to provide the desired reinforcement and/or repositioning clinical outcomes. FIGS. 3D and 3E show the engagement of the openings in the tensile members shown in FIGS. 3A and 3B to the anchor shown in FIG. 3C. Once the opening of the tensile member is placed over the end of the anchor, the anchor end firmly locks to the tensile member securing the tensile member to the anchor. As shown in FIGS. 3F to 3H, tensile members can be connected to anchors at the ends of the tensile members, at the mid-region of the tensile members, or anywhere where a suitable anchoring opening is located.

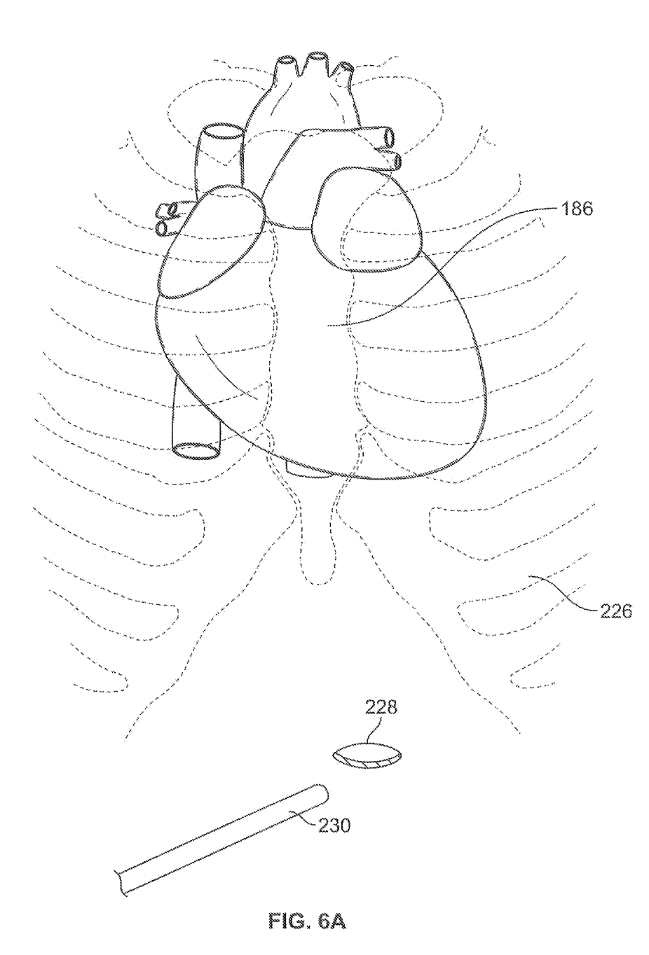

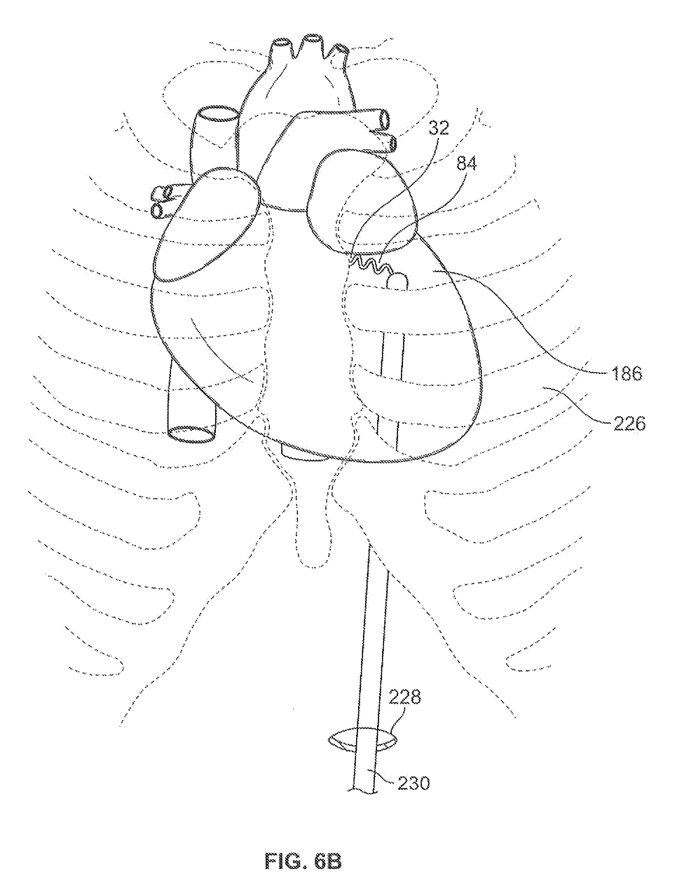

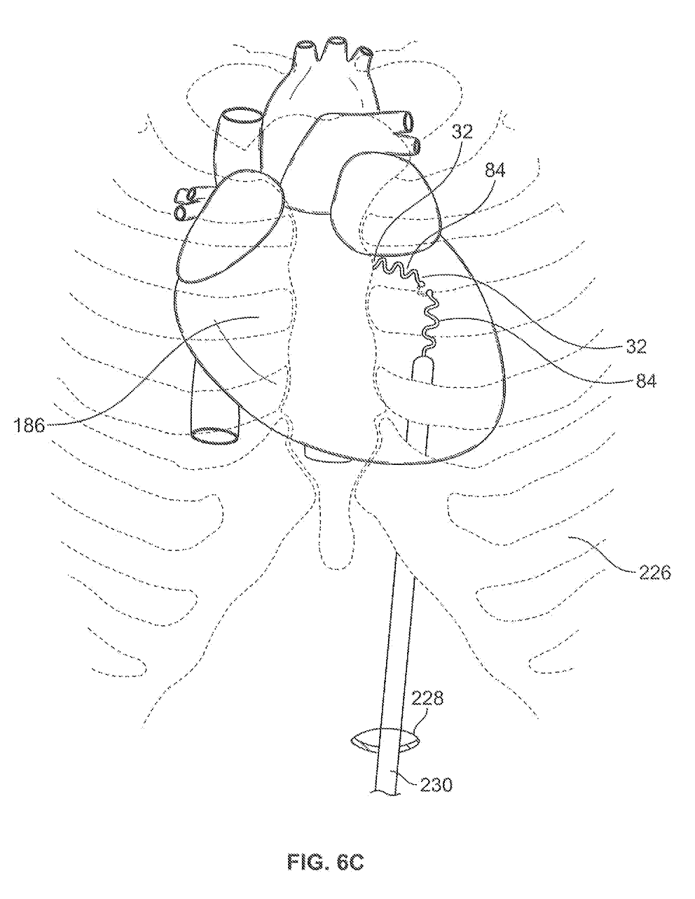

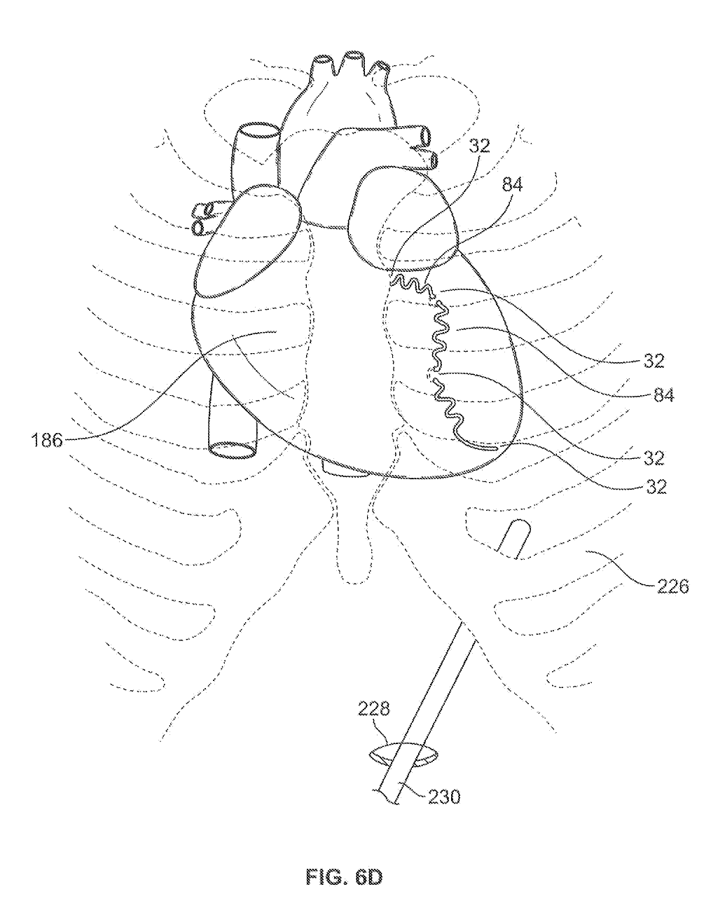

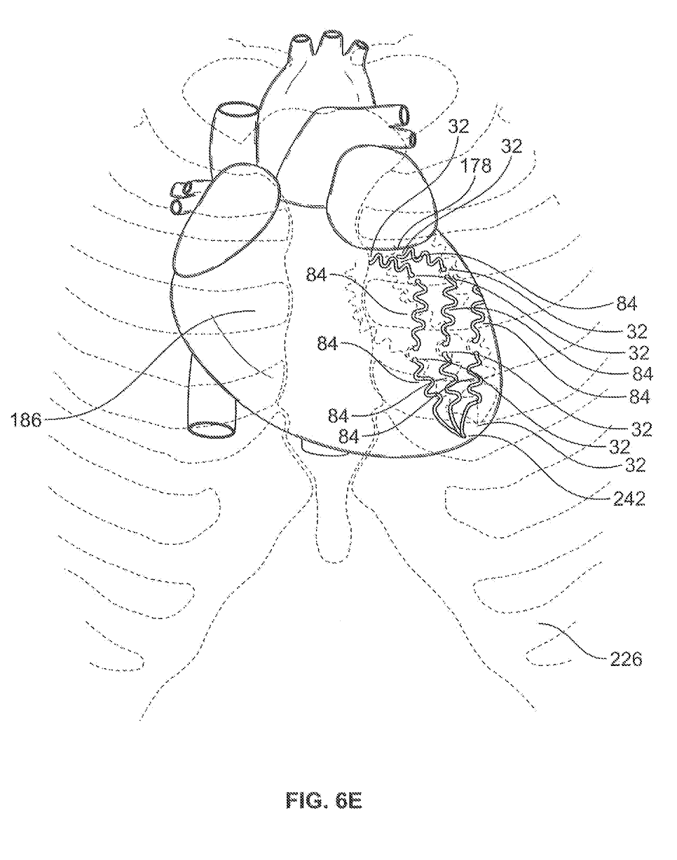

FIGS. 6A to 6D show a delivery system 230 capable of simultaneously and/or independently inserting components of a cardiac support structure 4 through or into myocardium via a sub-xiphoid 228 surgical approach. The discussion for this embodiment is described from a surgical approach initially inserting anchors for the cardiac support structures through the epicardium 68 to access the myocardium 34; although it should be noted that a catheter-based approach can be utilized with these embodiments if modified for percutaneous access and fluoroscopic visualization requirements facilitating insertion of the cardiac support structures either through the endocardial surface to access to or through the myocardium.

One embodiment of the cardiac support structure deployment system of the invention is provided as step-by-step illustrations showing initial delivery and positioning, followed by release and secured anchoring of the device upon the heart at the operator chosen anatomical locations in FIGS. 6A to 6D. The delivery system embodiment shown in FIGS. 6A to 6D involves a sheath capable of compressing the components (anchors and/or tensile members) of the cardiac support structure into a sufficiently low profile for placement typically through a trocar. The delivery system embodiment in FIGS. 6A and 6B also shows the components of the cardiac support structure compressed into a low profile inside a sheath having sufficient radial strength and column strength to straighten the tensioning structure 84 of support structure 4. To facilitate deployment, a stylette (not shown) may be positioned within the sheath to engages the free end of the anchor and/or tensile member 84 of the cardiac support structure. An operator advances or retracts the anchor and/or tensile member 84 as the stylette is advanced or retracted from a proximal end of the deployment device.

### Muscle Fiber Helix Restoring Cardiac Support Structures

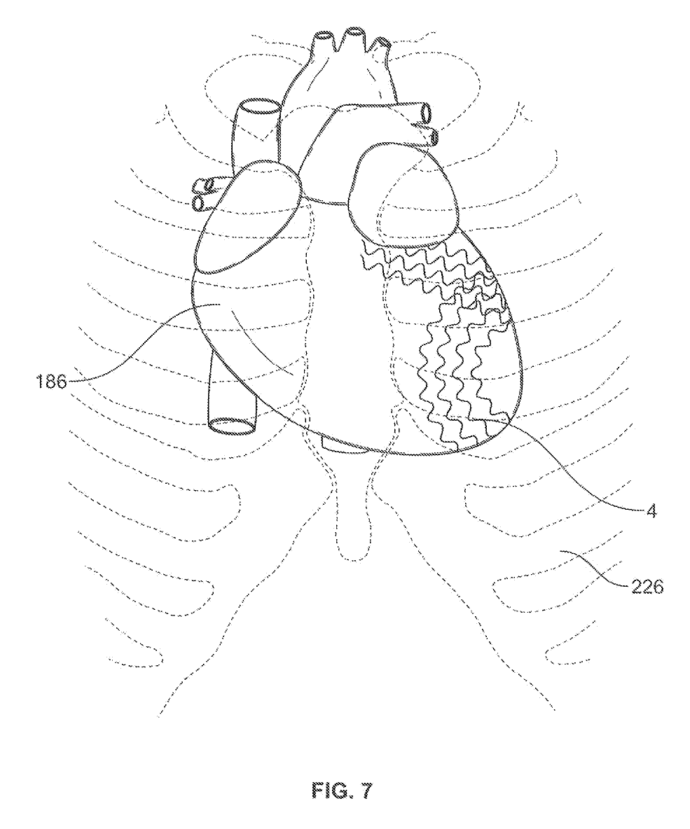

FIGS. 6E and 7 show perspective views of two 3-dimensional, helical, cinching, cardiac support structure 4 embodiments that utilize anchors 32 at each end of tensile members 84 to define the support structures 4. The tensile members 84 are anchored into a helical pattern from the atrial-ventricular groove 178 towards the apex 242. The cardiac support structure 4 embodiments in FIGS. 6E and 7 are positioned along the epicardium of the left ventricle and extend from the anterior surface to the posterior surface of the heart. Once positioned, the tensile members compress the heart wall into a helical orientation thereby maintaining or restoring the normal, helical pattern of the myocardial fibers.

Note that with the anchor 32/tensile member 84 embodiment of the invention, the cardiac support structures constructed can be configured into any pattern as determined by the operator during the implant procedure. One pattern is the desired helical pattern partially or substantially around the anterior and posterior surfaces of the left ventricle 18; others include partially or completely around the left and right ventricles (18 and 24), along the left ventricle 18 from the anterior surface to the posterior surface along the ventricular septum 244 and back to the anterior surface, or other configuration. In any case, the pattern will generally be one that follows or coordinates with the directionality of underlying heart muscle fiber orientation.

During deployment of tensile members whose anchoring mechanism involves inserting a loop of the cardiac support structure 4 through myocardium 34 (as shown in FIGS. 8, 9, and 10A), each free end of the tensile member 84 may be placed through a holder of a puncturing device where the puncturing device(s) are compressed inside a deployment sheath. In a minimally invasive surgical approach, it is preferred that the two puncturing devices are placed in contact with the epicardial surface (or alternatively can be placed into contact with the endocardial surface for catheter-based or open surgical procedures). The puncturing devices may be designed to penetrate the epicardium with sharpened or beveled tips at spaced apart intervals. Prior to inserting the puncturing devices, the tensile member can be placed through a pledget or other atraumatic surface (e.g., an ePTFE patch, polyester patch, other synthetic patch, a piece of pericardium, muscle or other tissue) to provide additional support at the anchor and to also provide additional strain relief to the underlying tissue once the tensile member is tightened. The puncturing devices are then typically advanced through the deployment sheath at which time they expand toward their preformed configuration channeling through myocardium to define a space for the tensile member to pass. Alternatively, the puncturing devices can pass the tensile member 84 from the epicardial surface through the myocardium, past the endocardium, along the endocardium, and back to the epicardium. Once the puncturing devices have advanced the ends of the tensile member through the heart wall and back past the epicardium, the ends of the tensile member will then be removed from the holder and the puncturing device is subsequently removed from the heart. The free ends of the tensile member are then tied or otherwise secured together thereby tightening and compressing a region of the heart wall. Again, prior to tightening the free ends of the tensile member, they can also be inserted through pledgets or other atraumatic structure to provide additional support and strain relief at the tissue puncture sites. For a more detailed description of the applicable procedure, including illustrations applicable to the noted hardware, reference is again made to co-pending U.S. Patent Application No. 2003/0078465, entitled, “Systems for Heart Treatment,” to Pai et al. incorporated herein by reference.

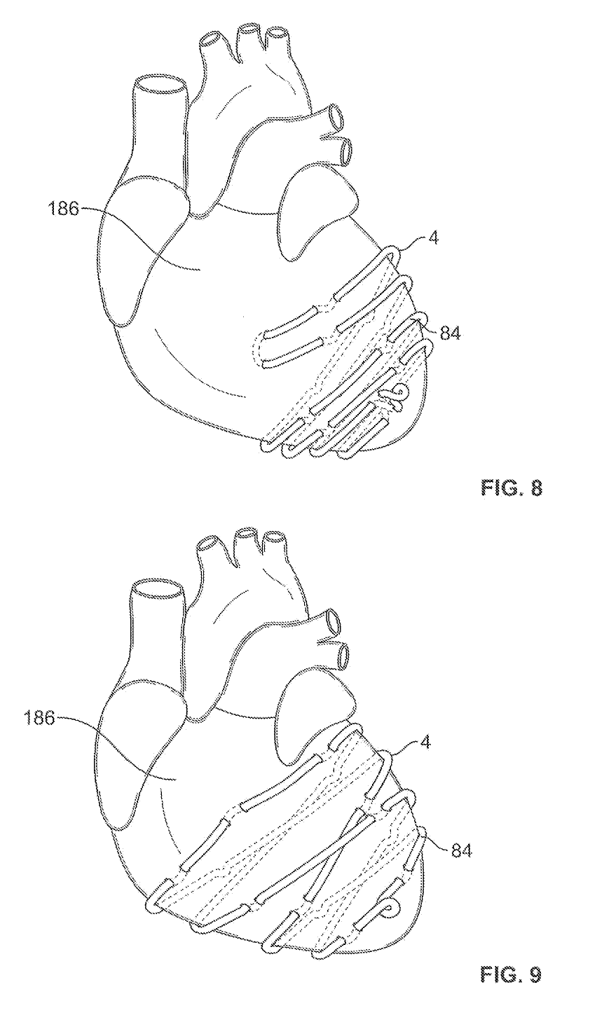

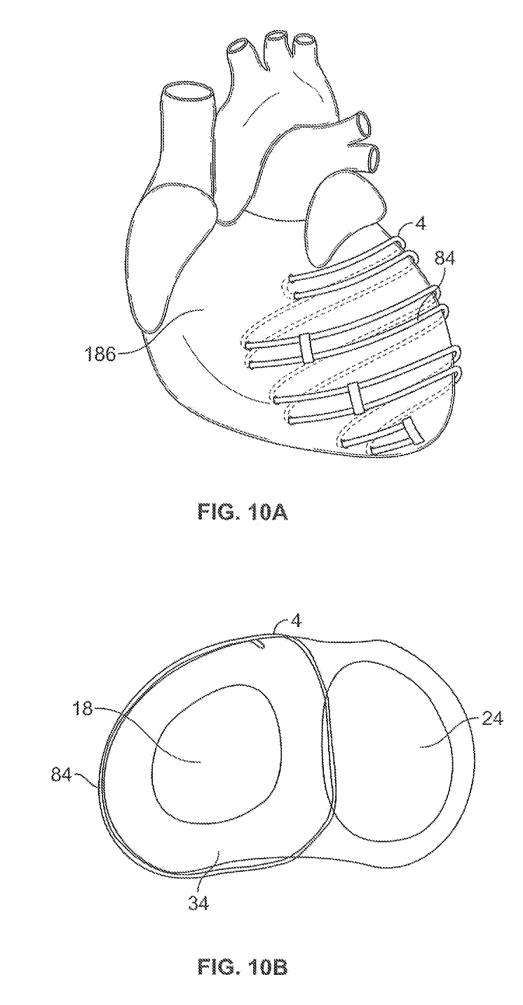

FIGS. 8, 9, 10A, and 10B show perspective views of hearts with cardiac support structures 4 placed through the myocardium 34 in helical patterns. In FIGS. 8, 9, and 10A, the solid lines demarcate the cardiac support structure located along the anterior surface of the heart, while broken lines demarcate the cardiac support structure looped into the myocardium and positioned along the posterior surface of the heart—both hidden from view. As shown, these cardiac support structure 4 embodiments pass through the myocardium along two spaced apart lines thereby producing a 3-dimensional helical cinching tensioning structure mechanism 4 capable of tightening/compressing the heart wall to urge or restore the helical myocardial fiber orientation. The tension applied to the heart by the cardiac support structures can be adjusted as required to alter the helical orientation of the myocardial fibers and impact wall motion. The helical pattern around the heart may be defined substantially only upon its surface as shown in FIGS. 8 and 9. In the alternative, the helical pattern may pass or carry through the heart to more selectively support a section of the heart such as the left ventricle as illustrated in FIGS. 10A and 10B.

It is additionally noted that the cardiac support structures can be oriented at or along other helical profiles relative to the heart thereby defining different tensioning patterns. The array of cardiac support structures previously discussed in reference to FIG. 5B illustrate one such alternative deployment system according to the present invention. In the embodiments of the invention in FIGS. 8, 9, 10A and 10B, a single tensile member 84 is shown deployed through the myocardium to define the support structure. In contrast, multiple cardiac support structures are positioned through heart tissue to produce a helical pattern in FIG. 5B to reposition the myocardial fibers into a helical pattern and restore a more optimal pattern of myocardial fiber contraction.

In any case, the cardiac support structures 4 of the invention can be positioned about the ventricles and anchored through or into myocardium so as to reposition previously relaxed, damaged or stretched myocardial fibers and restore their helical orientation. Restoring the helical myofibril orientation aids cardiac output by increasing the left ventricular ejection fraction and wall motion throughout the heart thereby improving efficiency and reducing the effects of congestive heart failure aiding the process of reverse remodeling.

### Papillary Muscle Repositioning

FIGS. 11A and 11B shows a representative three-dimensional, cinching, cardiac support structure 4 capable of repositioning and compressing the chordae tendonae 110 and/or the papillary muscles 174. These approaches may be employed in connection with the helical placement of support structures described above, on in isolation. A combined technique may be desire for many patients. Also, it should be appreciated that the teaching regarding papillary muscle repositioning taught in “Systems For Heart Treatment,” (2003/0078465) incorporated herein by reference and discussed above may be employed in connection with the additional teaching set forth herein.

In any case, according to the present invention, the papillary muscles may be preferentially repositioned relative to each other if these structures have migrated laterally due ventricular dilatation. Any pattern of cardiac support structures 4 can be used to provide the desired recovery or reverse remodeling response where the cardiac support structures extend between papillary muscles 174. By compressing the papillary muscles 174 together along the lateral free wall of the heart (or alternatively along the septal wall, not shown) the orientation of the valve leaflets and the chordae tendonae 110 are influenced. By reducing tension on the chordae tendonae 110 and valve leaflets exerted by over-stretched papillary muscles 174, valve leaflet apposition is improved thereby reducing mitral regurgitation and aiding reverse remodeling.

The flexibility of the cardiac support structures 4 enable the physician to custom tailor the treatment options to the patient after careful analysis of the valve competency, ventricular wall motion, ejection fraction, and other diagnostic parameters. The free ends of these three-dimensional, cinching, tensioning structures 4 can be tied together permanently or secured to a mechanism capable of twisting the knotted regions or otherwise manipulating the free ends to adjust or tighten the tensioning structures 4 intraoperatively, during a follow-up procedure, or remotely post procedure. Again, these adjustments can facilitate chronic maintenance of positive hemodynamic conditions.

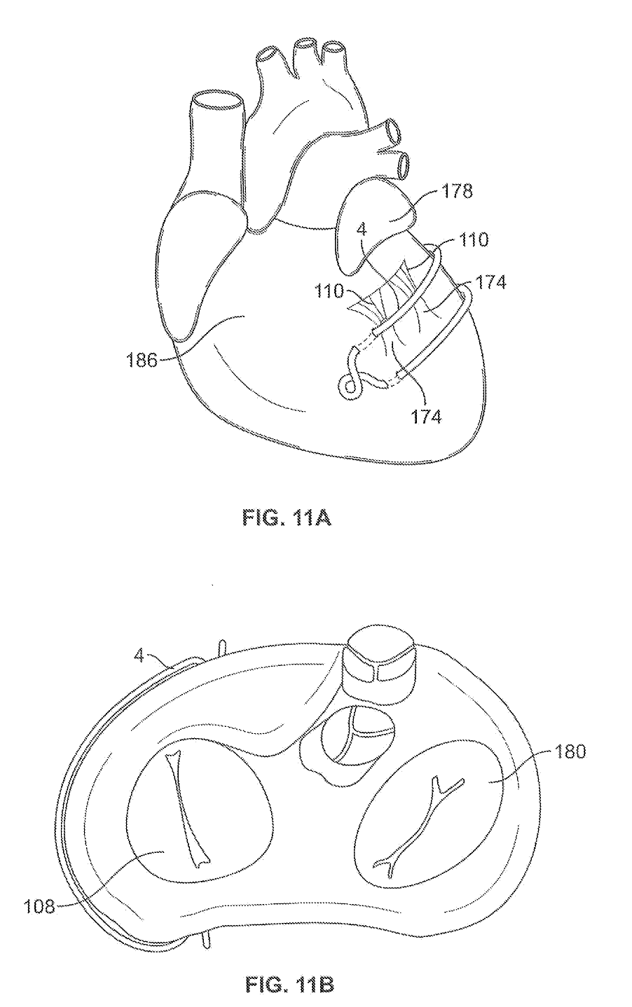

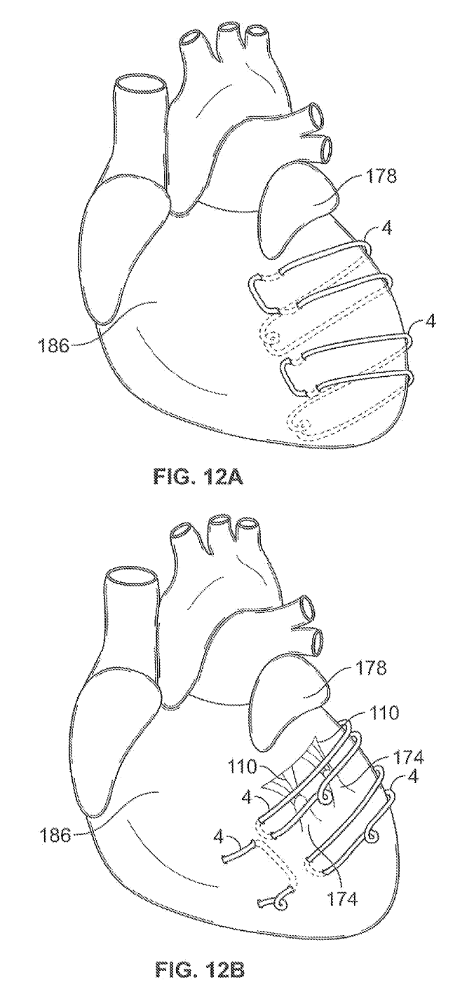

FIGS. 12A and 12B show alternative three-dimensional, cinching, cardiac support structure patterns 4 incorporating multiple tensile members 84 and anchors 32 to reposition the papillary muscles 174 relative to each other. In these cardiac support structure embodiments, the tightening force is distributed at more than one location (i.e., the insertion and knotted sites) thereby ensuring that a long, tightening structure will be capable of compressing tissue between the ends of the three-dimensional, cinching, tensioning structure 4 and repositioning the papillary muscles 174 closer together radially.

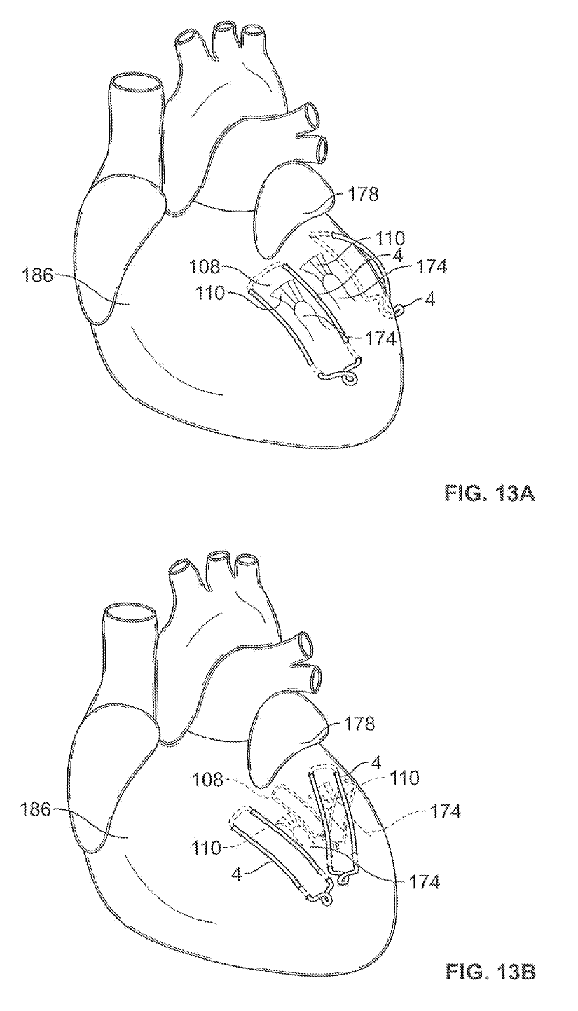

In FIGS. 13A and 13B, three-dimensional, cinching, cardiac support structures 4 are positioned and secured from the papillary muscles 174 to the atrial-ventricular groove 178 at the base of the heart. These cardiac support structure patterns reposition the papillary muscles 174 relative to the mitral valve annulus 108 thereby reducing tension placed on the chordae tendonae 110 and thereby reposition the valve leaflets for better apposition in order to reduce or eliminate mitral regurgitation.

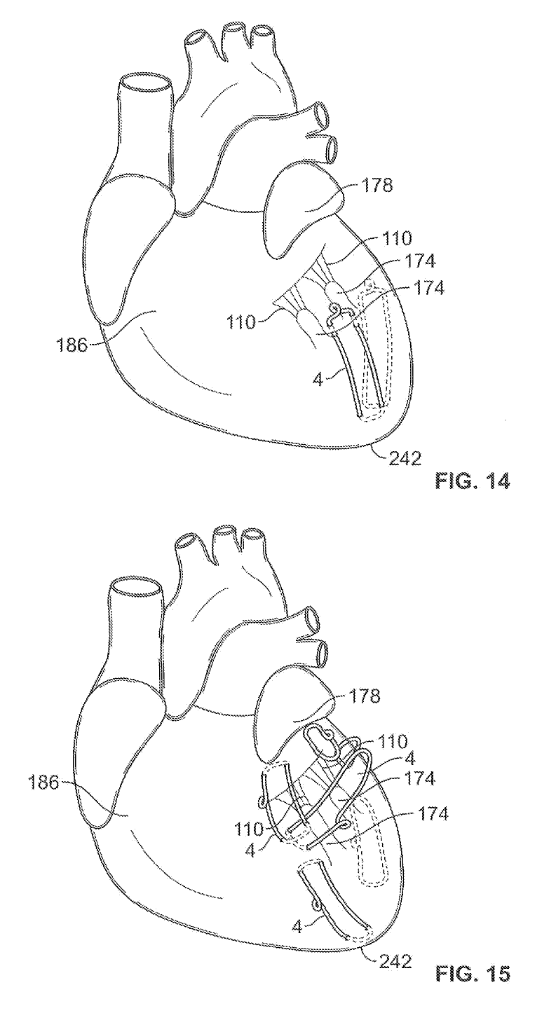

FIG. 14 shows a three-dimensional, cinching, cardiac support structure that repositions the apex 242 of the heart relative to the papillary muscles 174. Such cardiac support structure patterns reshape the apex of the heart relative to the papillary muscles 174 reinforcing the apical region of the heart and preventing excess wall tension from enlarging the apex 242 and also maintains the optimal location of the subvalvular apparati thereby also reducing or eliminating mitral regurgitation.

FIG. 15 shows a heart with multiple, independent three-dimensional, cinching, cardiac support structures 4 strategically positioned and secured between the papillary muscles 174 and other heart regions to reposition the papillary muscles 174 and reinforce the heart. In this embodiment, cardiac support structures are positioned between papillary muscles 174, from the papillary muscles 174 to the atrial-ventricular groove 178 at the base of the heart 186, and from the papillary muscles 174 to the apex 242. This configuration repositions the valve leaflets into more proximate apposition and reinforce the heart wall to encourage reverse remodeling.

### Active Cardiac Support Structures And Integrated Multi-Site Pacing

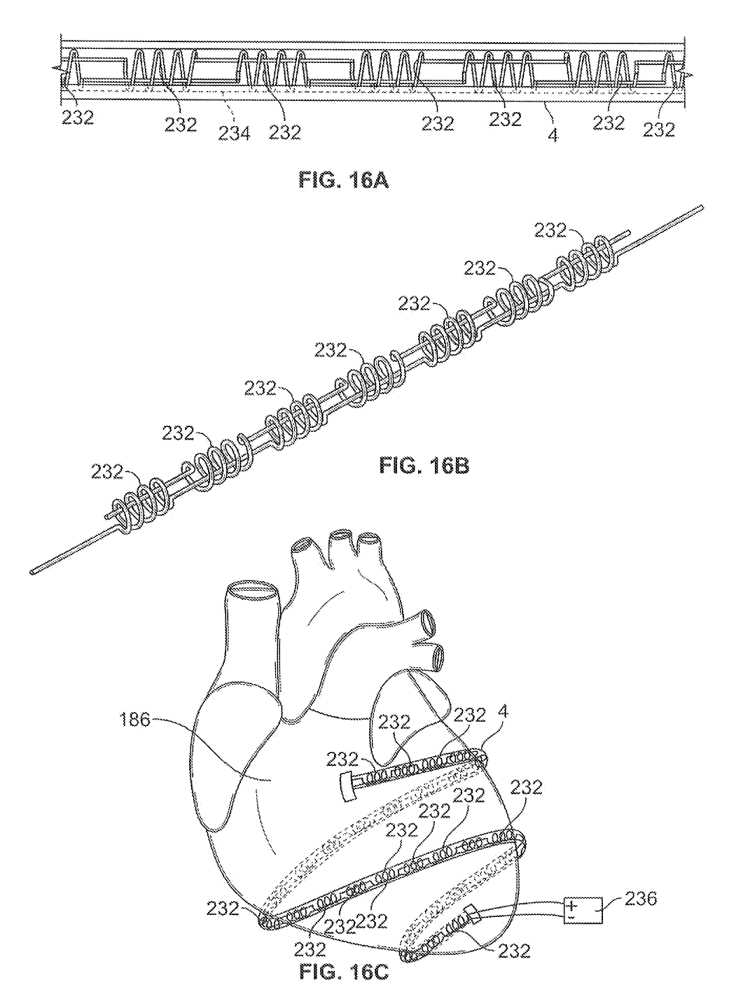

FIG. 16A shows a cardiac support structure 4 that incorporates multi-site pacing capabilities integrated into the cardiac support structure. As shown in FIG. 16B a set of two discrete wires are wound into spaced apart helical electrodes 232. Generally, wires fabricated from stainless steel, spring steel, titanium, titanium alloys, or other alloy may be wound in sections into one or more helices. The helical section(s) advantageously operate as spring member(s) as well as electrode(s).

A covering 234 encapsulates the wires and electrode(s) 232 exposing the electrode(s) through windows opposite at least a portion of the electrodes in the covering. Covering 234 (e.g., urethane, polyurethane, silicone, or other implantable polymer) may be extruded, injection molded, or dipped around the wire(s) such that discrete regions of the wires are exposed to define the electrode(s). Alternatively, laser cutting, chemical etching, or other removal process may be used to cut regions of covering to expose the electrode(s).

The embodiment shown in FIGS. 16A and 16B shows two discrete signal wires defining multiple electrodes 232 in the integrated cardiac support structure 4 to enable pacing (or electrogram recording) in bipolar mode between adjacent electrode pairs. FIG. 16C shows the cardiac support structure 4 with integrated multi-site pacing positioned and secured to the heart 186 in a helical pattern around the heart 186 thereby restoring the more physiologic myocardial fiber orientation and enabling pacing of the heart 186 at multiple sites along the heart 186 facilitating a patient specific, positive, inotropic response as required. The embodiment in FIG. 16C connects one signal wire thus the associated electrodes to the positive terminal on the implantable pacemaker 236 and the other signal wire to the negative terminal on the implantable pacemaker 236.

In other embodiments (not shown) where a single wire is used to define discrete electrodes, the pacing can be applied in unipolar mode from the electrodes to another reference electrode (e.g., the conductive cam of the pacemaker 236 or another electrode positioned within the body).

It should be noted that any combination of signal wire numbers, electrode numbers, electrode lengths, electrode diameters, and connection schemes can be used to tailor the integrated multi-site pacing lead and heart compression/reinforcement mechanism. Indeed, the synergistic combination of multi-site pacing and cardiac reinforcement offered by the subject structure (especially when configured for helical application to the heart) with an integrated support structure takes advantage of the benefits in contractility demonstrated with multi-site pacing adapted to the patient's specific needs and the mechanical compressing and reverse remodeling observed with tension reduction and volume reduction.

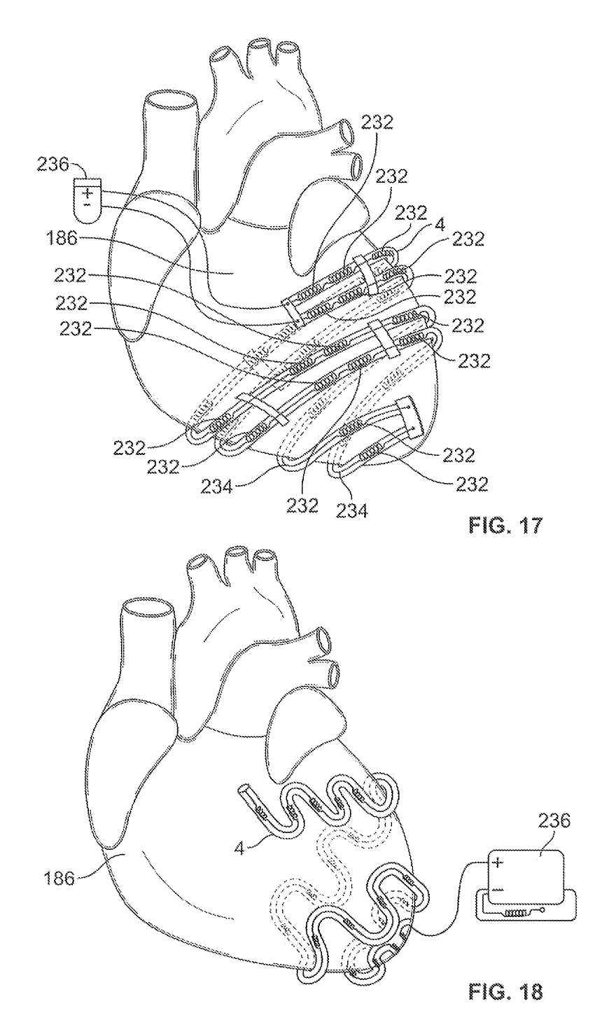

FIGS. 17 and 18 show alternative embodiments of cardiac support structures integrating multi-site pacing capabilities. In FIG. 17, separate cardiac support structures with integrated electrodes are placed in helical patterns spaced apart. Opposing structures are connected to either positive or negative terminals of the pacemaker 236 to induce a pacing pulse from electrodes on one structure to the electrodes on the adjacent structure. In FIG. 18, a single cardiac support structure integrating electrodes is wound around the heart 186 in a helical pattern and connected to a pacemaker that incorporates a cam electrode or is connected to a separate reference electrode (not shown) positioned in the body for the opposing terminal.

### Cardiac Support Structure With Integrated Active Compression Mechanism

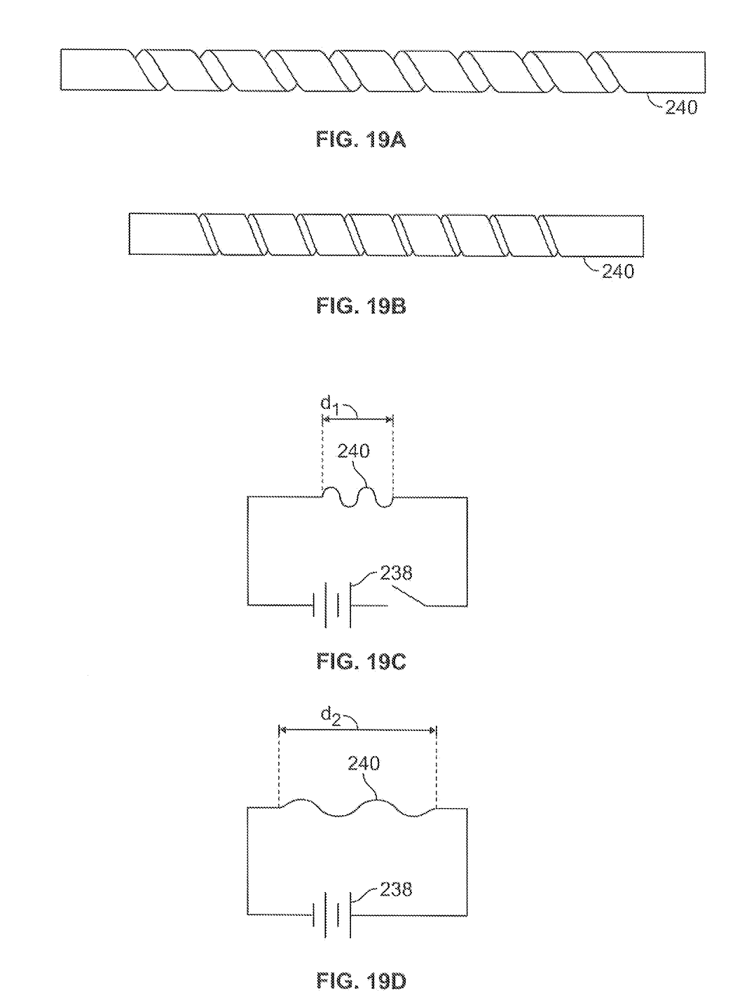

FIGS. 19A to 19D show a cardiac support structure embodiment tension element 84 (such as illustrated in FIGS. 3A and 3B) that can be caused to actively expand and/or compress in response to an energy source 238 such as through resistive heating (e.g., when using thermoelastic shape memory alloy materials) or via applied potential/current (e.g., when using piezoelectric materials or electroactive polymers).

Such active cardiac support structures could be arranged to work in synchrony with the requirements of the heart's a-v node, an implantable pacemaker 236 or any prescribed or desired requirement as driven by an energy source 238 specifically designed to work with the structure. In any case, FIGS. 19C and 19D show tensile member 84 in compressed and expanded states, respectively, with such action is driven by energy source 238.

As with cardiac support structures 4 employing multi-site pacing capabilities, the synergistic combination of active compression and cardiac reinforcement with an integrated support structures can be configured to provide a patient-specific active contractile assistance during systole while simultaneously providing the benefit of reverse remodeling observed with tension reduction and volume reduction. The structures can be configured to provide active contraction in synchrony with a pacemaker or similar controller to provide contraction as determined by the pacemaker circuitry algorithm or on demand as required.

### Structure Materials and General Fabrication Methods

The various embodiments of the invention will generally be fabricated from various biological, metallic, and/or polymeric materials as typically employed by those with skill in the art. Certain cardiac support structures comprise tensile members 84 (e.g., tube, ribbon, strand, or wire, which can limit elongation with satisfactory elasticity based upon the selection of material properties and cross sectional area) incorporating at least one stress distribution feature such that the tensioning structure 4 can apply tension against tissue without damaging the contacted tissue regions. A variety of materials can be used as the tensile member 84 of the tensioning structure 4, including PTFE, expanded PTFE, nylon, silicone, urethane derivatives, polyurethane, polypropylene, PET, polyester, superelastic materials (e.g., nickel titanium alloy), other alloys (e.g., stainless steel, titanium alloy etc.), metal (e.g., titanium), biological materials (e.g., strips of pericardium, collagen, elastin, vascular tissue such as a saphenous vein or radial artery, tendons, ligaments, skeletal muscle, submucosal tissue etc.) other alternate materials having the desired properties, or a combination of these and other materials.

The performance of the cardiac support structure will depend upon and can be tailored to the desired features. For example, when column strength is required, superelastic materials or other alloys or metals are preferred tensile member bodies 84 of the tensioning structure 4. When pure tension is required and the cardiac support structure is to be deployed through tortuous access points, more flexible materials such as expanded PTFE, polyester, or other suture type materials may be preferred as tensile members. When absorption or biological integration is desired over a period of time, biological materials such as strips of pericardium or collagen, or absorbable materials are preferred.

In instances where anchor members 32 are secured to one or more tensile member(s) 84, the anchors may be fabricated from biocompatible materials commonly used in medical implants including nickel titanium (especially, for self-expanding or thermally-actuated anchors), deformable stainless steel (especially for balloon-expanded anchors), spring stainless steel, or other metals and alloys capable of being deformed using balloon catheters or other expansive means, or self-expanded to secure the tensioning structure 4 to the vasculature, myocardium, or other tissue. Alternatively, the anchors 32 can be fabricated from superelastic polymers, flexible or deformable polymers such as urethane, expanded PTFE, or stiff materials such as FEP, polycarbonate, etc.

For self-expanding components of the embodiments (e.g., some tensile member embodiments), those components are preferably fabricated from a superelastic, shape memory material like nitinol (nickel titanium alloy). These types of materials elastically deform upon exposure to an external force and return to their preformed shape upon reduction or removal of the external force. Superelastic shape memory alloys enable straining of the material numerous times without plastic deformation. The repetitive strain capability facilitates a limited systolic stretch to enable adequate cardiac output while limiting or restricting the possibility of over stretch and continuation of the cyclic damage.

Various components of the cardiac support structures can be fabricated from shape memory alloys (e.g., nickel titanium) demonstrating stress-induced martensite at ambient temperature. Other shape memory alloys can be used and the superelastic material can alternatively exhibit austenite properties at ambient temperature. The composition of the shape memory alloy is preferably chosen to produce the finish and start martensite transformation temperatures (Mf and Ms) and the start and finish austenite transformation temperatures (As and Af) depending on the desired material response. When fabricating shape memory alloys that exhibit stress induced martensite the material composition is chosen such that the maximum temperature that the material exhibits stress-induced martensite properties (Md) is greater than Af and the range of temperatures between Af and Md covers the range of ambient temperatures to which the support members are exposed. When fabricating shape memory alloys that exhibit austenite properties and do not transform to martensite in response to stress, the material composition is chosen such that both Af and Md are less than the range of temperatures to which the supports are exposed. Of course, Af and Md can be chosen at any temperatures provided the shape memory alloy exhibits superelastic properties throughout the temperature range to which they are exposed.

By way of example, nickel titanium alloy having an atomic ratio of 51.2% Ni to 48.8% Ti exhibits an Af of approximately −20° C.; nickel titanium having an atomic ratio of 50% Ni to 50% Ti exhibits an Af of approximately 100° C. Melzer A, Pelton I. A. Superelastic Shape-Memory Technology of Nitinol in Medicine. Min Invas Ther & Allied Technol. 2000: 9 (2) 59-60. Such superelastic components are able to withstand strain as high as about 8 to 10% without plastically deforming.

Materials other than superelastic shape memory alloys can replace superelastic materials in appropriate cardiac support structure components provided they can be elastically deformed within the temperature, stress, and strain parameters required to maximize the elastic restoring force, thereby enabling the tensioning structures 4 to exert a directional force in response to an induced deflection. Such materials include other shape memory alloys, bulk metallic glasses, amorphous Beryllium, suitable ceramic compositions, spring stainless steel 17-7. Elgiloy™ and related alloys, superelastic polymers, etc.

The tensile members of various force transfer structure embodiments can be fabricated from at least one rod, wire, suture, strand, strip, band, bar, tube, sheet, ribbon or other such raw material having the desired pattern, cross sectional profile, dimensions, or a combination of cross-sections. These raw materials can be formed from various standard means including but not limited to: extrusion, injection molding, press-forging, rotary- forging, bar rolling, sheet rolling, cold drawing, cold rolling, using multiple cold working and annealing steps, or casting. When using superelastic materials or other alloys as the tensile members, they can be cut into the desired pattern and thermally formed into the desired three-dimensional geometric form. The tensile members can then be cut into the desired length, pattern or other geometric form using various means including, but not limited to, conventional abrasive sawing, water jet cutting, laser cutting, EDM machining, photochemical etching, or other etching tecliniques. The addition of holes, slots, notches and other cut away areas on the support structure body facilitates the capability to tailor the stiffness of the implant.

The tensile members, especially those that employ the use of tubular or wire raw materials, can also be further modified via centerless grinding means to enable tensile members that are tapered (i.e., have a cross-sectional diameter on the proximal end of the structure that progressively ramps down to a smaller cross-section on the opposite or distal end).

When fabricating superelastic tensile members from tubing, the raw material can have an oval, circular, rectangular, square, trapezoidal or other cross-sectional geometry capable of being cut into the desired pattern. After cutting the desired pattern, the tensile members are formed into the desired shape, heated, for example, between 300° C. and 600° C., and allowed to cool in the preformed geometry to set the shape of the tensile members.

When fabricating superelastic tensile members from flat sheets of raw material, the raw material can be configured with at least one width, W, and at least one wall thickness, T, throughout the raw material. As such, the raw sheet material can have a consistent wall thickness, a tapered thickness, or sections of varying thickness. The raw material is then cut into the desired pattern, and thermally shaped into the desired three-dimensional geometry. Opposite ends or intersections of thermally formed tensile members can be secured by using shrink tubing, applying adhesives, welding, soldering, mechanically engaging, utilizing another bonding means or a combination of these bonding methods. Opposite ends of the thermally formed tensile members can alternatively be free-floating to permit increased flexibility.

Once superelastic tensile members are fabricated and formed into the desired three-dimensional geometry, the supports can be electropolished, tumbled, sand blasted, chemically etched, ground, or otherwise treated to remove any edges and/or produce a smooth surface.

### CLAIMS

The previous discussions provide description of minimally invasive, cardiac support structures used to treat degenerative heart disease in patients suffering any stage of congestive heart failure. In addition, the described inventions provide methods and devices to provide restriction of continued enlargement of the heart, potentially progressively reducing heart size via reverse remodeling (i.e., application of compressive force during both systole and diastole), improving atrial pump synchrony and efficiently thereby mitigating the morbidity and mortality effects of atrial fibrillation and finally decreasing valvular regurgitation associated with said enlargement. However, those skilled in the art should appreciate that at least certain ones of the structures described herein can be applied across a broad spectrum of organ structures to provide reinforcement and to limit enlargement facilitated by compensatory physiologic mechanisms.

Accordingly, the invention is not to be limited to the uses noted or by way of the exemplary description provided herein. Numerous modifications and/or additions to the above-described embodiments may be applied; it is intended that the scope of the present inventions extend to all such modifications and/or additions. The breadth of the present invention is to be limited only by the literal or equitable scope of the following claims. That being said, we claim:

1. A method of treating valvular dysfunction of a heart including, the method comprising;
inserting a first end of a tensioning structure into myocardial tissue of the heart,
inserting a second end of a tensioning structure into myocardial tissue of the heart, and
tensioning and securing the tension of the tensioning structure to reposition at least one of the chordae tendonae or papillary muscles by compressing the papillary muscles together.

2. The method of claim 1, wherein the papillary muscles are compressed together along the lateral free wall or septal wall of the heart.

3. The method of claim 1, wherein the tensioning structure is inserted to be positioned substantially around the left ventricle of the heart.

4. The method of claim 1, wherein the tensioning structure is inserted to be positioned between the papillary muscles and the atrial-ventricular grove of the heart.

5. The method of claim 1, wherein the tensioning structure is inserted to be positioned between the papillary muscles and the apex of the heart.

6. The method of claim 1, wherein a plurality of tensioning structures are inserted, tensioned and secured in the myocardial tissue of the heart.

7. The method of claim 6, wherein placement of the tensioning structures includes at least two positions selected from those in claims 3-5.

8. A method of treating a heart, the method comprising;
inserting a portion of at least one cardiac support structure into the myocardial tissue, and
aligning another portion of the at least one support structure with the helical myofibril orientation of a portion of the heart, and
securing the position of the at least one support structure.

9. The method of claim 8, wherein the at least one support structure comprises at least one resilient tensile member and at least two anchors.

10. The method of claim 9, wherein only the anchors penetrate the myocardial tissue of the heart and the at least one resilient member lies along the surface of the heart.

11. The method of claim 9, wherein the at least one support structure comprises a plurality of adjacent tensile members.

12. The method of claim 9, wherein the tensile members are interconnected.

13. The method of claim 8, wherein a plurality of independent cardiac support structures are arranged in an array aligned with the helical myofibril orientation of a portion of the heart.

14. A method of treating a heart, the method comprising;
providing at least one resilient cardiac support structure aligned with the helical myofibril orientation of a portion of the heart, and
applying an electrical current for heart pacing via electrode portions of the structure.

15. A method of treating a heart, the method comprising;
providing at least one resilient cardiac support structure aligned with the helical myofibril orientation of a portion of the heart, and
applying an electrical energy to the cardiac support structure to cause it to provide active forcing assistance to the heart.

16. The method of claim 15, wherein the assistance is in expansion.

17. The method of claim 16, wherein the assistance is in contraction.

18. A system for treating the heart, the system comprising:
an array resilient elements positioned substantially along the helical myofibril orientation of a portion of the heart.

19. An apparatus for treating the heart, the apparatus comprising:
at least one resilient elongate member adapted to helically encircling at least a portion of a heart, the member including a plurality of electrodes adapted for multi-site pacing.

20. An apparatus for treating the heart, the apparatus comprising:
at least one resilient elongate member adapted to helically encircling at least a portion of a heart, the member including a plurality of actuators along a substantial length of the member to expand or contract upon application of energy.
